(19) United States
(12) Patent Application Publication
(10) Pub. No.: US 2024/0274277 A1
(43) Pub. Date: Aug. 15, 2024

(54) METHOD AND SYSTEM FOR CONNECTING INDIVIDUALS WITH MENTAL HEALTH SUPPORT BASED ON LANGUAGE AND CULTURAL BACKGROUND

(71) Applicant: PSYMOOD TECHNOLOGIES INC., Toronto (CA)

(72) Inventors: Rodrigo Skazufka BERGEL, Scarborough (CA); Frederico Guilherme NEUMANN, Toronto (CA)

(21) Appl. No.: 18/290,164

(22) PCT Filed: May 13, 2022
(86) PCT No.: PCT/CA2022/050764

§ 371 Date: Nov. 10, 2023

Related U.S. Application Data
(60) Provisional application No. 63/187,998, filed on May 13, 2021.

(51) Int. Cl. G16H 40/20; G16H 20/70

(52) U.S. Cl. CPC G16H 40/20; G16H 20/70

(57) ABSTRACT

A method and system for connecting individuals with mental health support based on language preference and cultural background. The system compares an individual's mental health support professional criteria with characteristics of mental health support professionals and then applies a weighting to the individual's mental health support processional criteria before determining a list of recommended mental health support professionals for the individual.

### CROSS-REFERENCE TO OTHER APPLICATIONS

The disclosure claims priority from U.S. Provisional Application No. 63/187,998 filed May 13, 2021 which is hereby incorporated by reference.

### FIELD

The disclosure is directed generally at the health care industry, and more specifically, at a system and method for connecting individuals with mental health support based on language and cultural background.

### BACKGROUND

Around 20% of people in the world suffer or have suffered from mental health issues in their lives. In some countries, 1 in 5 individuals experience a mental health problem or illness every year. With respect to immigrants and minorities, these numbers are even higher. Many issues such as language barriers, a lack of social support and not having a professional network in their new home country, as well as dealing with any kind of racial prejudice, makes it more challenging when seeking mental health support.

In recent years, these numbers are increasing tremendously, and mental health support is not only important but necessary. Being vocal about mental health issues is considered a taboo for many cultures. Furthermore, many immigrants find it difficult to express their troubles in a second (non-native) language to a stranger that does not understand their language or culture. The industry lacks the ability to understand and service this particular obstacle since each individual has unique mental health needs based on values and experiences.

Therefore, there is provided a system and method of connecting individuals with mental health support based on language and cultural background.

### SUMMARY

The disclosure is directed at a method and system for connecting individuals with mental health support based on language and cultural background. In one embodiment, the disclosure may be seen as a culturally sensitive digital platform designed to help individuals, such as, but not limited to, immigrants and minorities, obtain mental health support.

In one embodiment, the system and method of the disclosure connects users, or individuals, with mental health support professionals that share a similar cultural background and can help them in their own language. In another embodiment, the disclosure primarily considers cultural background, language, religion and cultural criteria when connecting individuals with mental health support professionals. In connecting the individuals with mental health support professionals, the disclosure may consider other factors or demographic information such as, but not limited to, geographical location, interests, and/or personal needs, to pair individuals with, or to recommend, at least one mental health professional for either online or in-person mental health support.

In one aspect, the disclosure provides individuals, more specifically, newcomers to a country, immigrants and/or minorities with an option to prioritize their mental health with suitable mental health support professionals. For example, minorities may face particular issues that only mental health support professionals or specialists who have already experienced the same issues in life could understand and be able to provide support, such as mental health support. Helping individuals address their concerns in a safe environment, as well as with someone they will feel more comfortable with—speaking in their language and having a similar cultural background may improve the individual's wellbeing.

In another embodiment, the disclosure is directed at a two-sided marketplace that matches individuals and mental health support professionals through input, such as answers to questions posed by the system. In one embodiment, the disclosure is directed at a platform that uses a culturally-sensitive algorithm that allows individuals to complete a questionnaire (which may be seen as an onboarding process), and then get matched with a mental health support professional in the language of their choice and based on the similarities in cultural background, among other factors. Examples of mental health support professional include, but not limited to, a psychotherapist, psychologist, or life coach.

In some embodiments, the system provides a list of recommended mental health support professionals based on the input from the individual to allow individuals to select a mental health support professional who understands their background and can counsel the individual to thrive and live a happier life. Individuals may also see videos, schedule sessions, participate in the session and pay using the method and system of the disclosure.

In another aspect of the disclosure, there is provided a method of connecting individuals for mental health support including receiving request from individual for connecting with a mental health support professional, the request including language preference and cultural background of the individual; comparing language preference with characteristics of a set of mental health support professionals; selecting a subset of the set of mental health support professionals that match the language preference of the individual; comparing cultural background of the individual with cultural background of the subset of the set of mental health support professionals; and generating and displaying a list of recommended mental health support professionals to the individual based on the comparisons.

In another aspect, the method includes receiving a selected mental health support professional from the individual based on the list of recommended mental health support professionals to the individual based on the comparisons. In yet another aspect, the method includes displaying a list of available times associated with the selected mental health support professional to the individual for selection of an appointment time. In a further aspect, the method includes transmitting video call information to the individual based on a selected appointment time. In yet a further aspect, the method includes comparing the mental health needs of the individual against the skill set and qualifications of the set of mental health support professionals. In another aspect, the method includes displaying a set of videos in different languages including at least the language preference, the set of videos associated with the list of recommended mental health support professionals. In yet another aspect, the method includes facilitating payment between the individual and the selected mental health support professional.

In another aspect of the disclosure, there is provided a method of connecting individuals for mental health support including receiving a request from individual for connecting with a mental health support professional, the request including a list of criteria for a desired mental health support professional, the criteria including language preference and cultural background of the individual; determining a list of recommended mental health support professionals for the individual based on the request; and displaying the list of recommended mental health support professionals.

In a further aspect, determining a list of recommended mental health support professionals includes generating a weighting for the list of criteria; comparing the list of criteria with characteristics of a set of mental health support professionals to determine which of the set of mental health support professionals meet the list of criteria; applying the weighting to the set of mental health support professionals that meet the list of criteria; and determining the list of recommended mental health support professionals based on which of the set of mental health support professional that meet the list of criteria receive a highest score based on the weighting. In another aspect, the method includes applying a distance(q,e)=(wf*sim(q,e)²)^{1/2} to the criteria where q represents a patient case, e represents a specialist case, f is an attribute of q and e, wf is the weighting factor and sim represents a similarity function. In yet another aspect, the list of criteria further includes type of treatment required.

In yet another aspect of the disclosure, there is provided a system for connecting an individual with mental health support including a registration module for collecting demographic information associated with the individual; an onboarding module for collecting mental health support professional criteria associated with the individual, the mental health support professional criteria including at least type of treatment, primary language and cultural background; and a decision module for determining a list of recommended mental health support professionals based on the mental health support professional criteria; wherein the decision module applies a weighting factor to the mental health support professional criteria when determining the list of recommended mental health support professionals.

In another aspect, the system includes a display module for generating or displaying content on a communication device associated with the individual. In yet another aspect, the system includes a communication module for communicating with a communication device associated with the individual. In a further aspect, the system includes a registration module for collecting demographic information associated with the individual. In yet a further aspect, the system includes a scheduling module for creating appointments between the individual and a selected mental health support professional. In an aspect, the system further includes a payment module for enabling payment between the individual and a selected mental health support processional after an appointment or session has been completed.

### DESCRIPTION OF THE DRAWINGS

Embodiments of the present disclosure will now be described, by way of example only, with reference to the attached Figures.

FIG. 1a is a schematic diagram of a system for connecting individuals with mental health support based on language and cultural background in its environment;

FIG. 1b is a schematic diagram of the system of FIG. 1a;

FIG. 1c is a schematic diagram of another embodiment of the system of the disclosure;

FIG. 1d is a schematic diagram of a back end module of the embodiment of FIG. 1c FIG. 2 is a flowchart outlining a method for connecting individuals with mental health support based on language and cultural background;

FIGS. 3a to 3h are example screen shots of questions displayed to an individual;

FIGS. 4a and 4b are further example screen shots;

FIG. 5 is a schematic diagram of an architecture of another embodiment of the system of the disclosure;

FIG. 6 is a screenshot of one embodiment of an administrative page showing matchmaking results;

FIG. 7 is a screenshot of one embodiment of a recommended list of mental health support professionals page; and

FIG. 8 is a table showing one embodiment of matchmaking calculations.

### DETAILED DESCRIPTION

The disclosure is directed at a method and system for connecting individuals with mental health support based on language and/or culture background. In one embodiment, the disclosure compares characteristics, or criteria, of the individual with characteristics of mental health support professionals to generate a list of those mental health support professionals that are best suited to work with the individual or a recommended list of mental health support professionals. This list may then be provided to the individual for selection of at least one of the mental health support professionals whereby the system and method of disclosure then connects the individual with the selected mental health support professional or professionals.

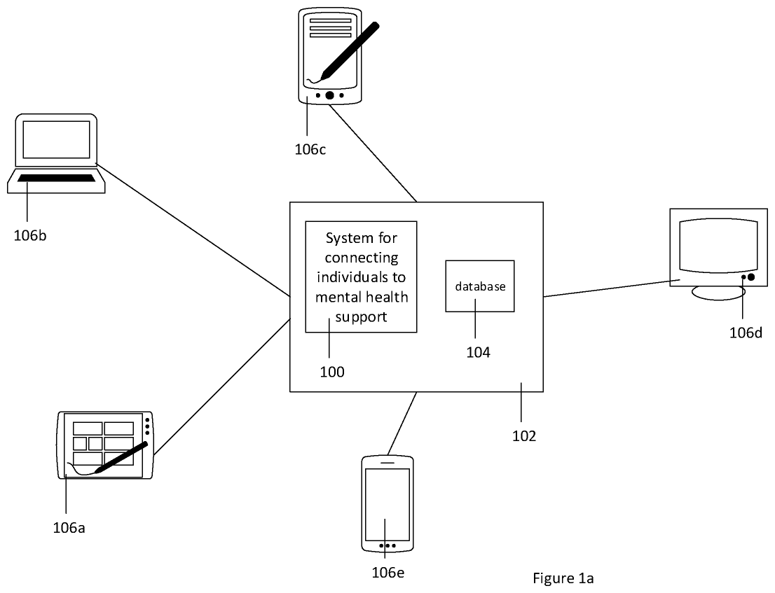

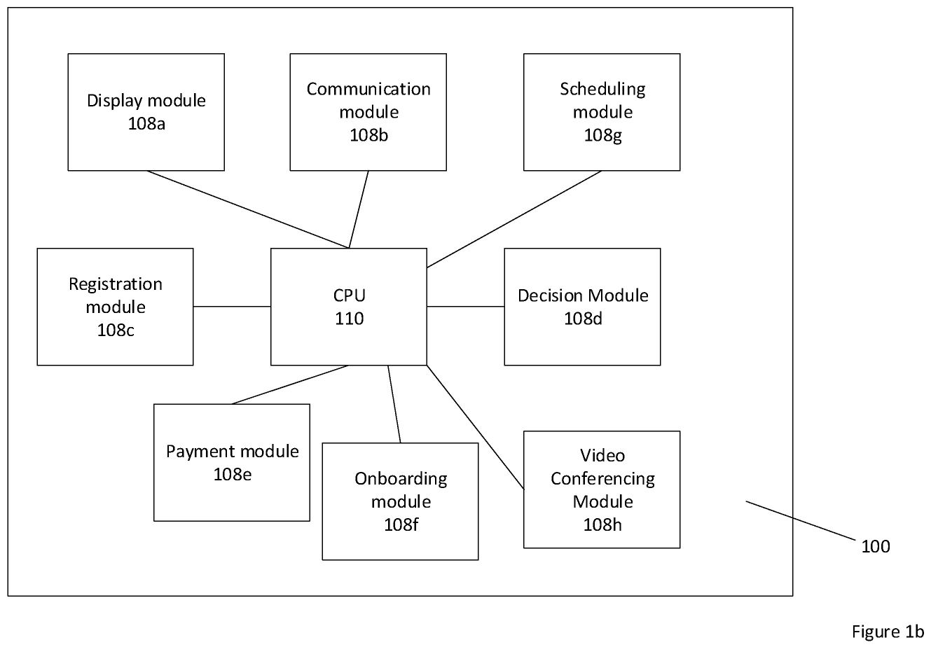

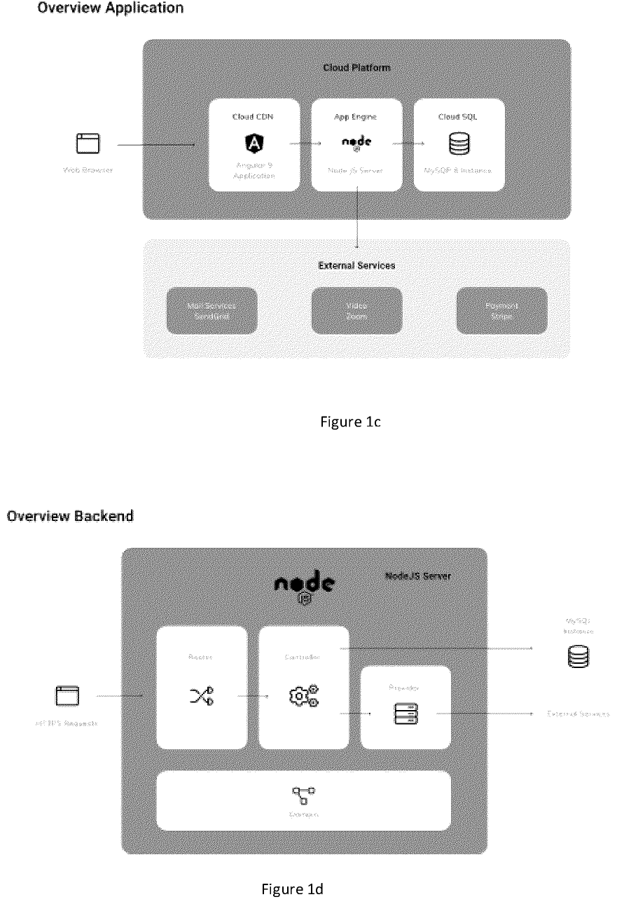

Turning to FIG. 1a, a schematic diagram of a system for connecting individuals with mental health support professionals based on language and cultural background is shown. The system 100 is stored within a processor, or server, 102 that is connected to, or may include, a database 104. The database 104 stores information relating to mental health support professionals and characteristics of these professionals such as, but not limited to, location of practice, years of experience, name, languages spoken, cultural background, capacity to take on new patients, hourly rate and areas of expertise. The database may also store information associated with individuals that has been previously input to the system.

Individuals may access the system 100 via different types of personal communication devices 106 such as, but not limited to, tablet 106a, laptop 106b, personal digital assistant (PDA) 106c, desktop computer 106d or smartphone/cellphone 106e. Communication between the personal communication devices and the system 100 will be understood.

Turning to FIG. 1b, a schematic diagram of the system is shown. In the current embodiment, the system 100 includes a plurality of modules 108 that perform different functionality to enable the method of connecting individuals with mental health support professionals based on language and cultural background.

The system 100 may include a display module 108a that generates the content or display page that is displayed by the system 100 on the personal communication devices 106 of the individuals. The display module 108a may determine the type of personal communication device 106 that the individual is using and then formats the display page accordingly. A communication module 108b may enable communication between the system 100 and the individual via the personal communication device 106. The communication module 108b may include software, hardware or firmware to enable the communication via known communication protocols.

The system 100 may further include a registration module 108c that enables individuals and/or mental health support professionals to register with the system 100. Registration of users with an online network or community will be well understood. The registration module 108c may also generate a set of questions for the individual to answer in order to retrieve specific or required information or criteria for selecting or determining a set of mental health support professionals that the individual may contact for an appointment. All of the information received by the registration module 108c may be stored in the database 104 or within a database associated with the system 100. A decision module 108d retrieves information associated with mental health support professionals and information or criteria associated with the individuals including language and cultural background information and then determines a list of mental health support professionals that best match the criteria or characteristics of the individual. The determination may include a weighting of the criteria submitted by the individual. The system 100 may also include a payment module 108e that performs the functionality of retrieving payment information (such as credit card, banking or PayPal™) associated with the individual and forwarding payment of invoices issued by the mental health support professional after an appointment has taken place.

Instead of being performed by the registration module, the system 100 may also include an onboarding module 108f that interacts with the individual to request specific or required information or criteria for selecting a set of mental health support professionals that the individual may contact for an appointment. A processor 110 controls each of the modules 108 to provide a central controller for the set of modules. The modules may communicate with the processor 110 or may communicate with each other in order to implement the method of the disclosure. The system may also include a scheduling module 108g for creating appointments between the individual and a selected mental health support professional. The system 100 may also include a video conference module 108h that enables an individual to use the system to have an online appointment or session with the mental health support professional.

Turning to FIG. 2, a method of one embodiment of connecting individuals with mental health support professionals based on language and cultural background is shown. It is assumed that the individual has registered with the system of the disclosure such as will be understood by those skilled in the art. Registration by the individual with the system (such as via the registration module) may include storing demographic information associated with the individual such as, but not limited to, name, address, sexual orientation, age, languages spoken, primary language, religion, place of birth, race, income, marital status, gender, cultural background and preferred communication language. Some or all of information, as well as the mental health issues or needs of the individual, is used to connect the individual, or patient, with a mental health support professional (of best fit) based on mental health support professional criteria, such as, but not limited to, the professional's primary language, age, religion, race, specialty, years of experience and rating and other data points collected by the system. In other embodiments, some or all of the information is used to prepare a recommended list of mental health support professionals for display to the user so that the user can select one or more mental health support professional to meet.

In one embodiment of registration, an individual may click on a sign up button that is provided on a login page associated with the disclosure. The individual may then choose to sign up as a patient whereby the system prompts the individual to fill out a form with personal information required. After submitting the form (which is received and stored by the system or registration module), the individual will receive a confirmation email from the system and with the account confirmed by the individual, the system enables or allows the individual to start the onboarding process. The onboarding process may be performed by the registration module or by the onboarding module. In some embodiments, the onboarding process may be performed after an individual requests an appointment as discussed below.

In the method of FIG. 2, the system receives a request from an individual to connect the individual with a mental health support professional (200). In one embodiment, the individual may log in to the system and then access a web page where the individual can request an appointment such as by clicking a “Request Appointment” button. The system may then request further detailed information relating to the type of mental health support professional that the individual wishes to meet with. In some embodiments, this information may be previously received as part of the onboarding process. In other embodiments, the system may request further information from the individual. Examples of different mental health professionals include, but are not limited to, a psychologist, a physchotherapist or a social worker. In other words, different specializations providing different services/kinds of therapy may be covered.

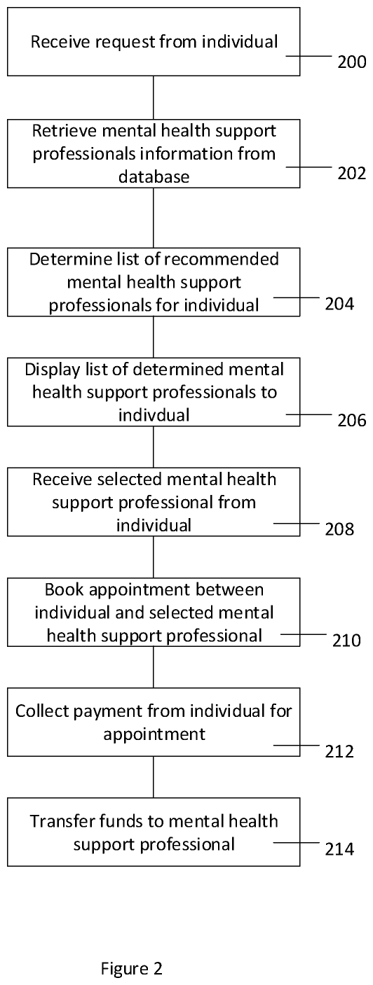

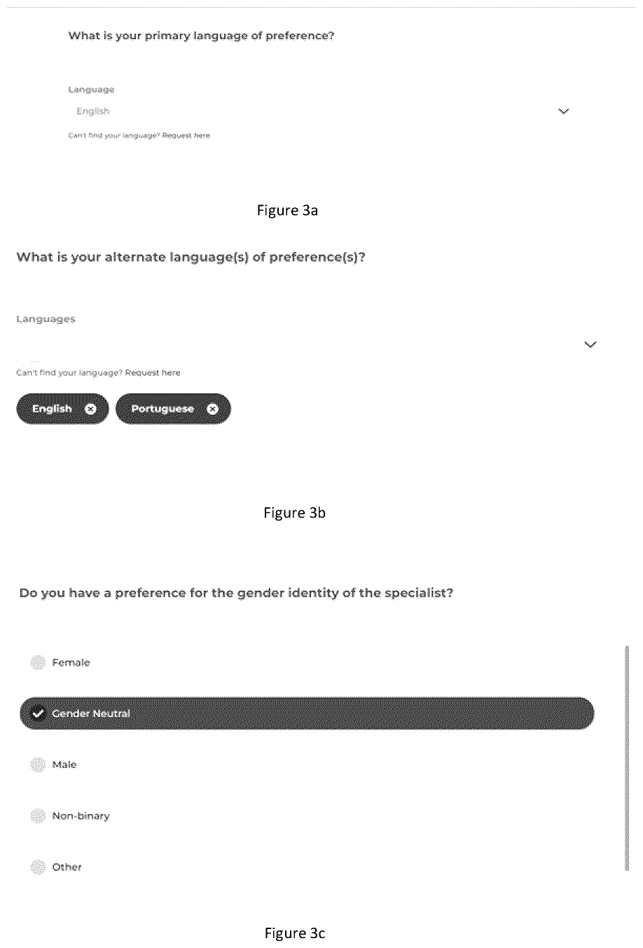

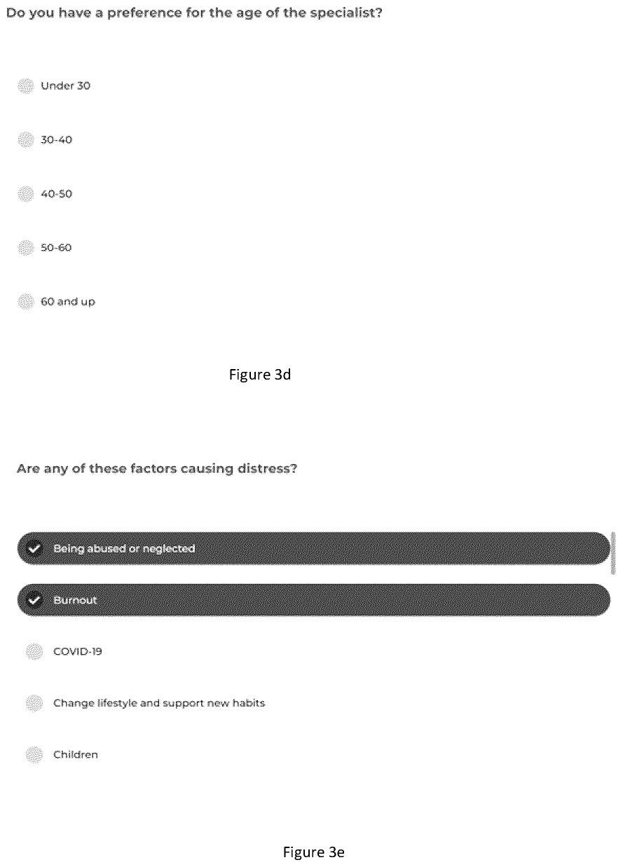

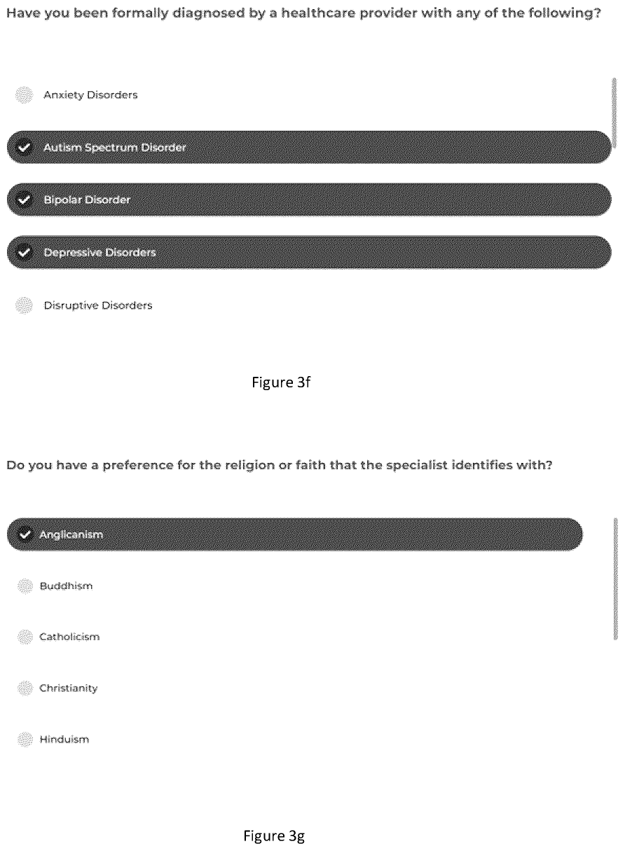

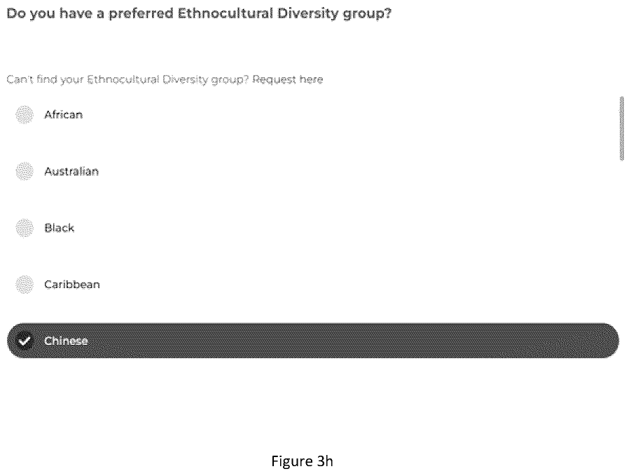

In another embodiment, the system may provide a list of questions for the individual to answer relating to the user preferences or mental health support professional criteria. This may also be performed in the registration process such that when a request is received, the system may retrieve the information relating to the list of questions from a database. In one specific embodiment, the list of questions may include eight (8) questions such as, but not limited to: “What is your primary language of preference?”, “What is your alternate language(s) of preference(s)”, “Do you have a preference for the gender identity of the specialists?”, “Do you have a preference for the age of the specialist?”, “Are any of these factors causing distress?”, “Have you been formally diagnosed by a healthcare provider with any of the following?”, “Do you have a preference for the religion or faith that the specialist identifies with?”, and “Do you have a preferred Ethnocultural Diversity group?”. FIGS. 3a to 3h provide example screenshots of the questions that may be asked by the system. In other embodiments, a different set of questions may be asked but which relate to criteria for the system to determine suitable mental health support professional for meeting with the individual.

After receiving all of the information required to fulfill the request, the system then retrieves all the mental health support professionals information from the database (202). Based on the information submitted by the individual, the system then generates a list of recommended mental health support professionals (204) based on the search criteria. As discussed above, the search criteria includes language and cultural background of the individual such that the list of recommended mental health support professionals speak the language and have an identical or similar cultural background to the individual.

In one embodiment, the system may retrieve an initial list of mental health support professionals from the database that speak the language of the individual requesting the appointment. The system may then compare the cultural background of the individual with the cultural background of the mental health support professionals in the list to determine which professionals match this criteria. The system continues to reduce the number of candidates for the recommend list of mental health support professionals based on predetermined search criteria until the number of professionals in the list equals or is less than the predetermined number.

In another embodiment, the system may applying a weighting system to the criteria being searched/compared. For example, the onboarding questions (such as the ones listed above) may be weighted in terms of importance. In one example, the criteria may be weighted as follows: criteria such as primary language, secondary languages, preferences, factors that cause distress and previously diagnosed conditions may be provided the same weighting factor; religion, age range and gender may be assigned the same weighting factor; and religion may be in its own category. All the weights, or weighting factors described above can be adjusted at any time such that in other embodiments, other criteria may be weighted more heavily.

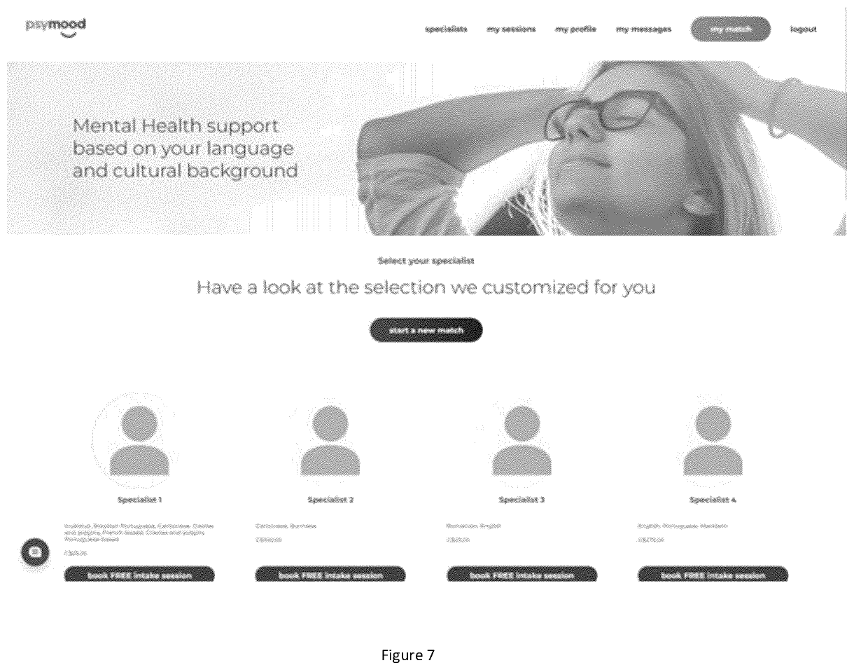

This recommended list of mental health support professionals is then displayed to the individual (206). An example screenshot is shown in FIG. 7. The list may be accompanied with information about each of the mental health support professionals so that the individual can learn a bit more about each professional in the list before making their decision with respect to the professional they wish to meet with. In some embodiments, the list of recommended mental health support professionals may include options to schedule an appointment or a free intake, send a direct message to the mental health support professional and/or an ability to see a profile of the mental health support professional. The system may also provide videos about or by the mental health support professional that is in a preferred language of the individual.

The individual may then select a mental health support professional from the list which is received by the system (208). The system then provides the functionality for the individual to book an appointment with the selected mental health support professional (210). In some embodiments, this is achieved by the system providing a list of available times for selection by the individual and then booking an appointment when receiving a selection from the individual. Alternatively, the system may retrieve a calendar associated with the individual and the mental health support professional and providing a list of commonly available times to the individual. In yet another embodiment, the individual may be prompted to input available times and the system then compares this with available time slots for the selected mental health support professional or professionals. In another embodiment, the system provides contact information to the individual to book their own appointment which may then be input to the system for tracking.

In some embodiment, the system may provide a platform for the individual to meet with the mental health support professional by integrating with a video call platform to facilitate the appointment between the individual and the support professional once an appointment has been set up. Examples of video call platforms include but are not limited to, Microsoft Teams™, Zoom™ or Google Meet™. Once an appointment is set up or booked, the system may then transmit video call information to the individual and the mental health support professional which may be in the form of an email or a calendar invite. This functionality may be provided by a video conference module.

If necessary, the system of the disclosure may also handle payment between the individual and the mental health support professional after the session is completed by collecting payment from the individual (212) and then transferring the funds to the appropriate mental health support professional (214).

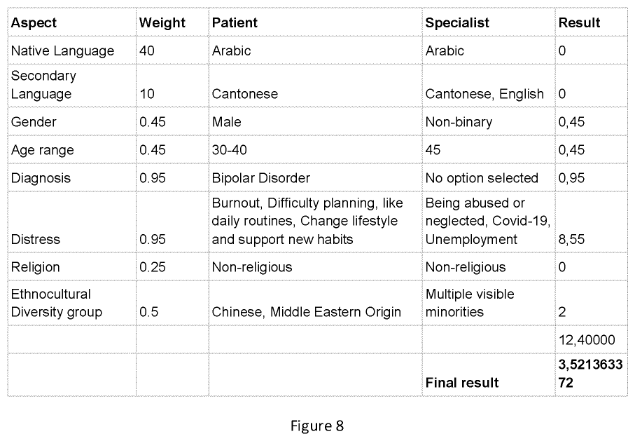

In one example method of determination, the criteria (which may be seen as individual aspects) are provided with a weighting factor that reflects the importance of that criteria. One example is shown in the table of FIG. 8. As shown, a “Native Language” aspect is assigned a weighting of 40, a “Secondary Language” aspect is assigned a weighting of 10, a “Gender” aspect is assigned a weighting of 0.45, an “Age Range” aspect is assigned a weighting of 0.45, a “Diagnosis” aspect is assigned a weighting of 0.95, a “Distress” aspect is assigned a weighting of 0.95, a “Religion” aspect is assigned a weighting of 0.25 and an “Ethnocultural Diversity group” aspect is assigned a weighting of 0.5.

After receiving the criteria from the individual associated with each of these aspects, the system compares the criteria with the characteristics of each of the set of mental health support professionals that are stored in the database. In the current example, the criteria for the individual is shown under the Patient column and the characteristics of the mental health support professional is shown in the Specialist column. After comparing the patient and specialist column, a score or result may be assigned for each aspect.

The score may be calculated using an algorithm such as distance(q,e)=(wf*sim(q,e)²)^{1/2}. One example calculation is as follows:

distance=√(40*(0²)+10*(0²)+0.45*(1²)+0.45*(1²)+0.95*(1²)+0.95*(3²)+0.25(0²)+0.5*(2²)

In another embodiment, the system may also store notes from the mental health support professional from the session or appointment that may be used for future matching or determination performed by the system between the individual and other mental health support professionals. This may be necessary if the previously selected mental health support professional is not available or suited for the needs of the individual as needs may change over time and from session or meeting to session or meeting.

In further embodiments, the system may provide different display pages to the individual such as, but not limited to, a listing of all mental health support professionals, a listing of all scheduled and completed appointments (where a user may cancel, rate, reschedule or schedule a new appointment), a profile page showing information associated with the individual and a messages page where the individual may send and receive messages from mental health support professionals.

For a mental health support professional interacting with the system, the mental health professional will also be required to initially register with the system, such as via the registration module. After registration, and onboarding, the mental health support professional may then interact with the system to meet with individuals or to have appointments scheduled with individuals. The system may also provide different display pages to the mental health professional such as, but not limited to, a profile page where the mental health support professional can enter their credentials, create an introductory video, input relevant license or certificate data and other personal information, a schedule page where the mental health support professional can see their schedule and a messages page where the mental health support professional may communicate with others in the system such as individuals or other mental health support professionals.

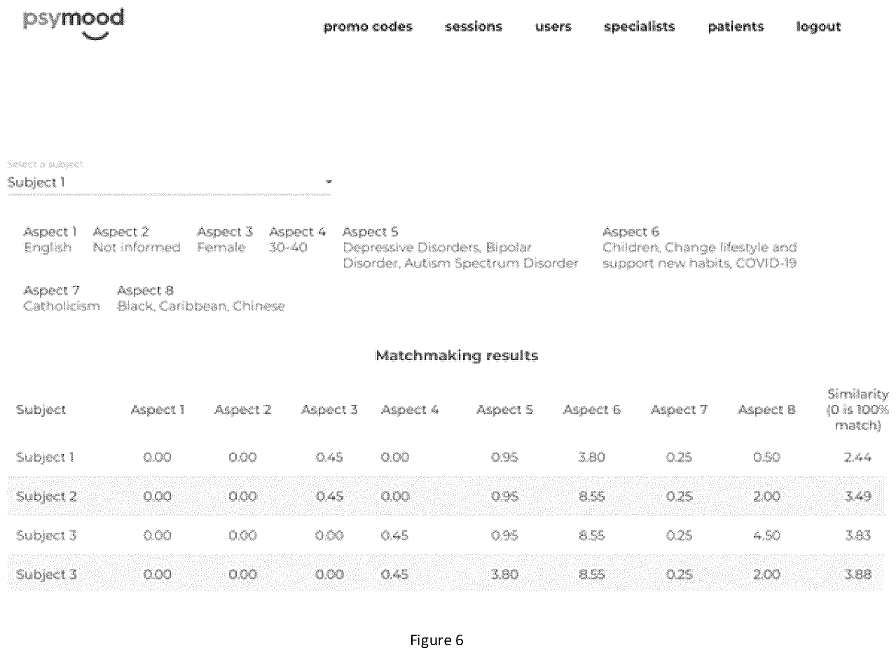

The system may further include an administration component for a user to interact with the system to include updates, provide promotions, checking mental health support professional ratings (based on input from individuals), remove individuals of mental health support professionals from the system, review personal information that has been entered by individuals or mental health support professionals. The system may also provide a display page showing analytical results from how the system determines a recommended list of mental health support professionals for an individual. An example matchmaking page is shown in FIG. 6.

FIG. 6 shows a set of aspects or criteria that are being compared based on the individuals input (such as during the onboarding process). The matchmaking results between the individual and a set of mental health support professionals (listed as Subjects in the current figure) are shown with a score or result listed. A similarity score (which may be seen as the final score as described with respect to FIG. 8 above) is listed in the matchmaking tables showing which mental health support professional is most similar to the individual. Individual scores with respect to the compared aspects may also be displayed.

Use of subjects may allow the mental health support professionals remain anonymous so that the system or administrator may see analytical information rather than personal information. In the current embodiment, a similarity score closer to 0 represents between match between the individual and the mental health support professional.

In one embodiment, the disclosure may be seen as a system and/or method that may be implemented as a web application that is implemented or integrated in a browser such as, but not limited to, Chrome™, Firefox™ or Edge™.

In another specific embodiment, when stored within the server, the system 100 (schematically shown in FIG. 1c), may include two separately running modules: Angular for a front end module and NodeJS for a back end module. The front end module may be seen as the module that interacts with external parties such as the individuals, the mental health support professionals and/or any other third parties that access the system while the back end module performs all the activities or functionality that does not require interaction with these external parties. The system 100 may also include another module that hosts a database, such as, but not limited to, a MySQL database.

When an individual interacts with the system, such as by browsing a web application, the Angular module renders all frontend components and provides an interface between the individual and the system. All data shown on the front end (or displayed to the individual or mental health support professional) is transmitted by the back end and database modules. Each data request that is received by the system or the back end module is processed by a router layer and is sent to the controller or processor. The processor is responsible for the business rules, processing every request, consulting the database and returning an answer. The answer may be the name of a mental health support professional or an individual, a schedule, the available time slots and the like. In one embodiment, database queries are performed using ORM to manipulate data. When external data is needed, the provider layer is responsible for those requests. In one specific embodiment, payments are handled by the Stripe™ platform and video calls supported by the Zoom™ video conferencing platform.

The back end module also determines the list of mental health support professionals that are suggested by to the individual. In one embodiment, the first criteria for a match between an individual and a mental health support professional, therapist or specialist may be by their main and secondary languages. After this filter, the system may then generate an ordered list by similarity. In one embodiment, the matching algorithm that is used by the system is expansion-ready and can analyse multiple signals, with specific weights, or weighting factors, such that a best match determination may be made. In some embodiments, the algorithm use inputs such as, but not limited to, religion, previous diagnoses, patient mood, sexual orientation and relationships in its determination. In another embodiment, the system may be executed on the Medstack Platform which is a cloud-based server management system. In another specific embodiment, the system may be hosted on Google Cloud, with code stored GitHub and Stripe and PayPal as a financial platform. An overview of one embodiment of a back end module is shown in FIG. 1d.

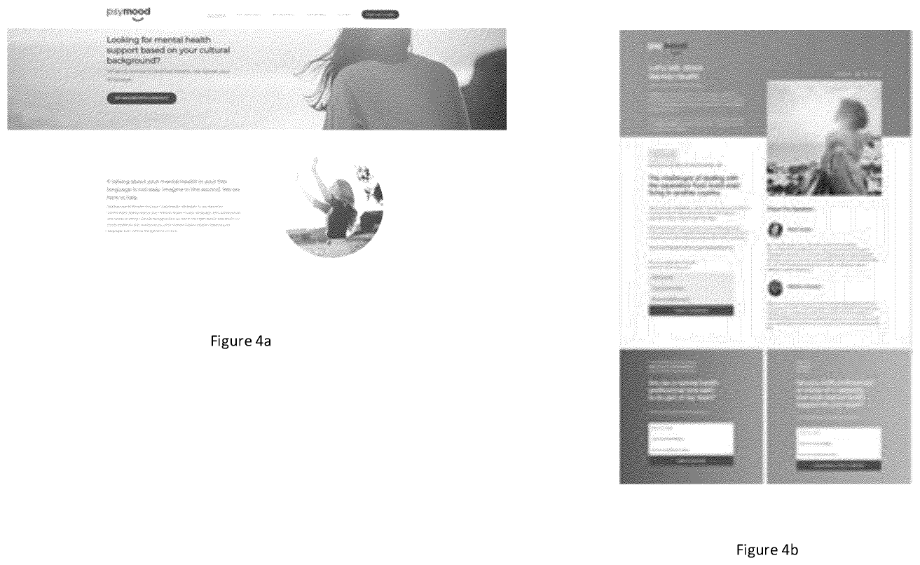

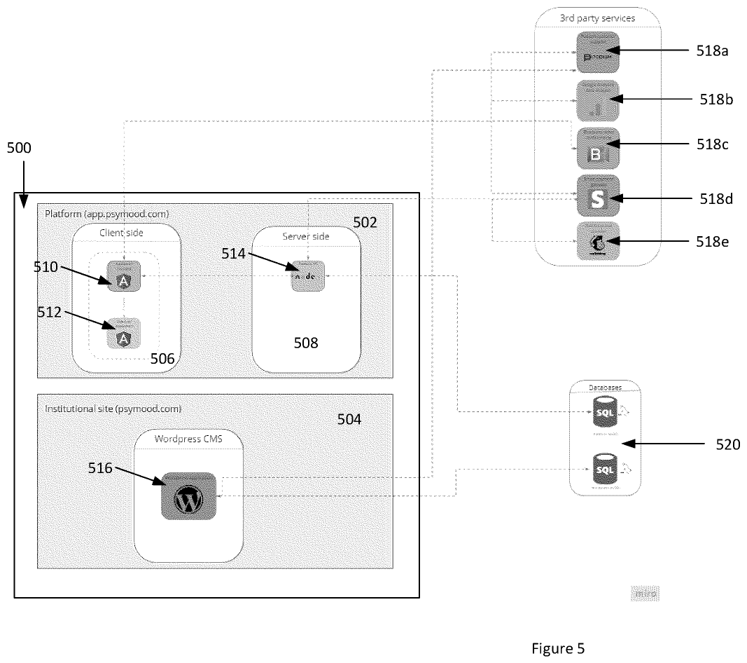

In another embodiment, the disclosure may be seen as a system and method that connects individuals to mental health professionals based on their first language and their cultural background to enable an individual to receive online therapy, counselling and/or coaching sessions, using a holistic approach to better help the individual deal with stress, anxiety, depression, and improve their overall well-being by connecting them with mental health professionals that may best fit their needs. Example screen shots that may be displayed to a user are shown in FIGS. 4a and 4b.

Turning to FIG. 5, a schematic diagram of an architecture of another embodiment of the system of the disclosure is shown. The system 500 is integrated within a server and operates as a platform for connecting individuals with mental health support based on language and cultural background. The system 500 may include a platform portion 502 and an institutional site portion 504. The platform portion 502 includes a client side component 506 and a server side component 508. In one embodiment, the client side component 506 may be seen as a front end component or module (such as discussed above) and the server side component 508 may be seen as a back end component or module (such as discussed above).

In this embodiment, the client side component 506 includes an application front end component 510 and a video call component 512. The application front end component 510 interacts with the user to request and receive information from the individual and to interact with the mental health support professional. Examples of information include but are not limited to, registration information including language preference and cultural background, name, address and the like. The front end component 510 may also request and receive onboarding information from the individual. The front end component may also receive payment information from the individual. The video call component 512 provides the functionality to connect the individual and the mental health support professional for a virtual face-to-face appointment at the scheduled time. The server side component 508 includes a platform API component 514 that is in communication with the application front end component 510 to store information provided by the application component 510 and to transmit requested information to the component 510 so that it may be transmitted by the system 500 to the individual or the mental health support professional.

The system may also communicate with third-party services 518 such as, but not limited to, a customer support service 518a, an analytics service 518b, a video conferencing service 518c, a payment gateway service 518d and a mailing service 518e. The institutional site component 504 may include a content management system (CMS) component 516. The CMS component 516 may store and provide educational information to individuals who may be interested in learning more about mental health or to learn more about different ways to treat mental health issues that do not involve a mental health support professional. The CMS component may also include further information for review by an individual.

The system may further be in communication with databases 520 to retrieve previously stored information or to store new information when received.

In one embodiment, the system may include some of the following functionalities a matchmaking functionality, a flexible pricing functionality, an exclusive content functionality, a scheduling and secure payment functionality, a secure videoconferencing functionality and a customization and packages for organization functionality.

The matchmaking functionality may be provided by amassing or registering a multidisciplinary team of mental health support professionals that are able to offer a solution for each individual based on the needs of the individual. One advantage of the disclosure is the holistic approach of having multiple kinds of professionals that can help individuals in their first language and based on cultural background. By connecting individuals with mental health support professionals who speak their first language and have a similar cultural background, the disclosure assists patients (or individuals) to feel supported and comfortable when working through their issues. In addition to language and cultural background, the matchmaking functionality may take other factors such as, but not limited to, therapeutic needs, wellness goals, and cost into consideration when pairing an individual with a mental health support professional. The disclosure enables individuals (in a virtual environment) to discuss their concerns with a mental health support professional from the comfort of their own space. These services can benefit clients adjusting their lives to a new environment, whether they have moved to a different country for a job opportunity, for health and safety, or to study as part of a foreign exchange program.

The flexible prices functionality provides flexible pricing that allows individuals to sign up for sessions beginning at a predetermined cost. In one embodiment, each mental health support professional knows how much their sessions should cost, and each individual knows how much they can and want to pay. In one embodiment, being a part of the mental health support professionals associated with the system may include a monthly subscription fee for these professionals. Individuals may use the system for free, only needing to pay for the sessions. One advantage of the system is that it also takes financial budgeting into consideration when pairing an individual with a mental health professional, so refugees or immigrants who may not have a promised source of income are able to find the best mental health services that suits their needs.

The exclusive content functionality provides exclusive content, such as in a blog section, through social media channels and YouTube, related to mental health for newcomers and minorities, in order to support their wellbeing. This exclusive content functionality is free and openly available online, even when individuals are not “paying” for therapy, counseling or coaching sessions. They still have resources at their disposal to help them in their mental health journeys, with weekly tips, monthly webinars, articles, videos, etc.

The scheduling and secure payment functionality enables individuals to schedule sessions, pay for it and receive the link in their email, all in a secure environment, without leaving the platform or system and is compliant with federals regulations.

The secure video conferencing functionality enables an individual to schedule the date and time of sessions through the system. Mental health support professionals have the freedom of indicating availability and hours of service and the individuals are able to book sessions based on the convenience of their own schedules. Once the session is booked, both the mental health support professional and individual will receive a secure link that allows them to virtually complete their session. By providing the secure video conference functionality, there is flexibility in choosing sessions, reduction in overall time commitment due to the lack of a commute, as well as the opportunity to complete the session in the individuals' own home.

The customization and packages for organizations functionality enables companies to facilitate a healthier and happier workplace, by providing resources to their employees (or the individuals) to support their mental health and well-being. One of the approaches of the disclosure is to offer inclusive and personal mental health services based on language and cultural background preferences to support the diverse nature or organizations' employee bases and promote inclusivity of employee assistance.

In one specific embodiment, all our data is compliant, respecting medical and privacy regulations and stored with MedStack.

In another embodiment, when an individual registers, the individual is prompted, such as by the registration module or the onboarding module, to answer questions about their wellness goals, cultural background and language preferences. This information will provide the system with the information or data to match individuals with mental health support professionals that share their language and cultural background. In one embodiment, the system displays to the individual a list of the mental health support professionals ranked by similarities and may be provided with videos of each mental health support professional (for viewing) in up to three different languages that they speak, to get to know more about them. Once an individual selects their preferred mental health support professional, the individual can complete their appointment booking and schedule their first session.

In one embodiment, in order to assist individuals, the first intake session may be free and the individual starts to pay if they choose the mental health support professional after the first intake session and after feeling confident with their choice. If they are not satisfied with their first choice, they may take a new free intake session with a different mental health support professional from the recommended list.

The disclosure may be seen as a 2-sided market place with mental health support professionals from all over the world that can offer flexible prices. One advantage of the system is the connecting of mental health support professionals and individuals from all over the world. Another advantage is that there are benefits for individuals to stay connected to their cultural background and having access through the disclosure provides access to individuals to mental health support professionals located anywhere in the world.

Other advantages include access for individuals to mental health support professionals from different nationalities and cultural backgrounds, who can provide services in different languages to better assist individuals' needs. When an individual accesses the system with a specific mental health need, the individual may be connected to a suitable mental health support professional who speaks his/her own language and has a similar cultural background. To be able to find a meaningful connection with a mental health support professional is a great deal if an individual can share his/her thoughts without the need of translating them into a secondary language, or trying to explain too much for someone who has not experienced something similar, which would already block them from properly expressing themselves, and could be even more threatening to their mental health.

In the preceding description, for purposes of explanation, numerous details are set forth in order to provide a thorough understanding of the embodiments. However, it will be apparent to one skilled in the art that these specific details may not be required. In other instances, well-known structures may be shown in block diagram form in order not to obscure the understanding. For example, specific details are not provided as to whether aspects of the embodiments described herein are implemented as a software routine, hardware circuit, firmware, or a combination thereof.

Embodiments of the disclosure or portions/aspects thereof may be represented as a computer program product stored in a machine-readable medium (also referred to as a computer-readable medium, a processor-readable medium, or a computer usable medium having a computer-readable program code embodied therein). The machine-readable medium can be any suitable tangible, non-transitory medium, including magnetic, optical, or electrical storage medium including a diskette, compact disk read only memory (CD-ROM), memory device (volatile or non-volatile), or similar storage mechanism. The machine-readable medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the machine-readable medium. The instructions stored on the machine-readable medium can be executed by a processor or other suitable processing device, and can interface with circuitry to perform the described tasks.

The above-described embodiments are intended to be examples only. Alterations, modifications and variations can be effected to the particular embodiments by those of skill in the art without departing from the scope, which is defined solely by the claims appended hereto.

What is claimed is:

1. A method of connecting individuals for mental health support comprising:
receiving request from individual for connecting with a mental health support professional, the request including language preference and cultural background of the individual;
comparing language preference with characteristics of a set of mental health support professionals;
selecting a subset of the set of mental health support professionals that match the language preference of the individual;
comparing cultural background of the individual with cultural background of the subset of the set of mental health support professionals; and
generating and displaying a list of recommended mental health support professionals to the individual based on the comparisons.

2. The method of claim 1 further comprising receiving a selected mental health support professional from the individual based on the list of recommended mental health support professionals to the individual based on the comparisons.

3. The method of claim 2 further comprising displaying a list of available times associated with the selected mental health support professional to the individual for selection of an appointment time.

4. The method of claim 3 further comprising transmitting video call information to the individual based on a selected appointment time.

5. The method of claim 1 further comprising comparing the mental health needs of the individual against the skill set and qualifications of the set of mental health support professionals.

6. The method of claim 1 further comprising displaying a set of videos in different languages including at least the language preference, the set of videos associated with the list of recommended mental health support professionals.

7. The method of claim 4 further comprising facilitating payment between the individual and the selected mental health support professional.

8. A method of connecting individuals for mental health support comprising:
receiving a request from individual for connecting with a mental health support professional, the request including a list of criteria for a desired mental health support professional, the criteria including language preference and cultural background of the individual;
determining a list of recommended mental health support professionals for the individual based on the request; and
displaying the list of recommended mental health support professionals.

9. The method of claim 8 wherein determining a list of recommended mental health support professionals comprises:
generating a weighting for the list of criteria;
comparing the list of criteria with characteristics of a set of mental health support professionals to determine which of the set of mental health support professionals meet the list of criteria;
applying the weighting to the set of mental health support professionals that meet the list of criteria; and
determining the list of recommended mental health support professionals based on which of the set of mental health support professional that meet the list of criteria receive a highest score based on the weighting.

10. The method of claim 8 further comprising:
applying a distance(q,e)=(wf*sim(q,e)²)^{1/2} to the criteria where
q represents a patient case, e represents a specialist case, f is an attribute of q and e, wf is the weighting factor and sim represents a similarity function.

11. The method of claim 8 wherein the list of criteria further comprises type of treatment required.

12. A system for connecting an individual with mental health support comprising:
a registration module for collecting demographic information associated with the individual;
an onboarding module for collecting mental health support professional criteria associated with the individual, the mental health support professional criteria including at least type of treatment, primary language and cultural background; and
a decision module for determining a list of recommended mental health support professionals based on the mental health support professional criteria;
wherein the decision module applies a weighting factor to the mental health support professional criteria when determining the list of recommended mental health support professionals.

13. The system of claim 12 further comprising a display module for generating or displaying content on a communication device associated with the individual.

14. The system of claim 12 further comprising a communication module for communicating with a communication device associated with the individual.

15. The system of claim 12 further comprising a registration module for collecting demographic information associated with the individual.

16. The system of claim 12 further comprising a scheduling module for creating appointments between the individual and a selected mental health support professional.

17. The system of claim 12 further comprising a payment module for enabling payment between the individual and a selected mental health support processional after an appointment or session has been completed.
